(19) United States
(12) Patent Application Publication
(10) Pub. No.: US 2007/0020654 A1
(43) Pub. Date: Jan. 25, 2007

(54) Methods and kits for preparing nucleic acid samples

(75) Inventors: John E. Blume, Sunnyvale, CA (US); Yanxiang Cao, Mountain View, CA (US); Kyle B. Cole, Stanford, CA (US); Kai Wu, Mountain View, CA (US); Charles G. Miyada, San Jose, CA (US)

Correspondence Address: AFFYMETRIX, INC;ATTN: CHIEF IP COUNSEL, LEGAL DEPT., 3420 CENTRAL EXPRESSWAY, SANTA CLARA, CA, 95051, US

(73) Assignee: Affymetrix, INC., Santa Clara, CA (US)

(21) Appl. No.: 11/419,459

(22) Filed: May 19, 2006

Related U.S. Application Data
(60) Provisional application No. 60/683,127, filed on May 19, 2005.

(51) Int. Cl. C12Q 1/68; C12P 19/34

(52) U.S. Cl. 435/6; 435/91.2

(57) ABSTRACT

The present invention provides methods for preparing nucleic acid samples. The methods of the present invention are particularly amenable for preparing samples that substantially represent the whole transcripts. In some aspects the methods include a step of reducing the amount of ribosomal RNA in a total RNA sample prior to amplification. In preferred aspects single stranded sense strand cDNA is generated, labeled and hybridized to arrays of probes. The method is particularly suitable to use with microarray based expression analysis.

### RELATED APPLICATIONS

This application claims the priority of U.S. Provisional Application No. 60/683,127, filed on May 19, 2005, the entire disclosure of which is incorporated herein by reference in its entirety.

### BACKGROUND OF THE INVENTION

Nucleic acid sample preparation methods have greatly transformed laboratory research that utilize molecular biology and recombinant DNA techniques and have also impacted the fields of diagnostics, forensics, nucleic acid analysis and gene expression monitoring, to name a few. There remains a need in the art for methods that amplify substantially entire transcripts.

### SUMMARY OF THE INVENTION

In one aspect of the invention, methods for preparing nucleic acid samples that represent RNA transcripts are provided. The methods are particularly suitable for preparing samples that are used for detecting transcript features such as exons and alternative splicing. The methods are suitable for quantitative, semi-quantitative or qualitative detection of such transcript features. The methods can be used to monitor a large number of transcripts including all types of variants such as alternative spliced transcripts. The methods are particular suitable for microarray based parallel analysis of a large number of, such as more than 1000, 5000, 10,000, or 50,000 different target transcripts or transcript features. As used herein, the term “target transcript” or “target nucleic acid” is used to refer to transcripts or other nucleic acids of interest.

In a preferred embodiment, the method for preparing a nucleic acid sample includes hybridizing a primer mixture with a plurality of RNA transcripts or nucleic acids derived from the RNA transcripts and synthesizing first strand cDNAs complementary to the RNA transcripts and second strand cDNAs complementary to the first strand cDNAs, where the primer mixture contains oligonucleotides with a promoter region and a random sequence primer region; and transcribing RNA initiated from the promoter region to produce the nucleic acid sample. The primer region can be a random hexamer. The promoter is typically a prokaryotic promoter such as a bacteriophage promoter, preferably a T7, T3 or SP6 promoter.

In a particularly preferred method, the resulting cRNA can be used as templates to synthesize second cDNAs. The second cDNA synthesis may be carried out using random primers such as random hexamer. In one embodiment, the second cDNAs are synthesized in presence of one modified DNA precursor nucleotide such as dUTP that is a substrate for Uracil DNA glycosylase. cDNAs are fragmented by excising the modified base with the UDG to generate abasic sites and cleaving at the abasic sites by means of an endonuclease, such as endonuclease IV or Ape I. A typical ratio of dUTP to dTTP is 1 to 3. dUTP can be incorporated into ss-cDNA during reverse transcription or into ds-cDNA during second strand cDNA synthesis. dUTP can be incorporated in a single strand such as the sense strand or the antisense strand or in both strands.

While the methods of the invention have broad applications and are not limited to any particular detection methods, they are particularly suitable for detecting a large number of, such as more than 1000, 5000, 10,000, or 50,000 different transcript features. For example, the second cDNAs may be fragmented and labeled and then hybridized with nucleic acids for detection. The labeling steps may be carried out, for example, during cDNA synthesis. Oligonucleotide probes are particularly suitable for detecting specific transcript features such as specific exons or splice junctions in transcripts. Typically, a collection of at least 5,000, 10,000, 50,000, 100,000 or 500,000 oligonucleotide probes may be used for detection. The nucleic acid probes may be immobilized on a collection of beads, on a single substrate or on several substrates, for example a two chip set.

In another aspect of the invention, a reagent kit for preparing nucleic acid samples is provided. An exemplary reagent kit contains a container comprising an oligonucleotide mixture component and instructions for use of the oligonucleotide mixture where the oligonucleotide in the oligonucleotide mixture component comprises a random primer region and a promoter region. One illustrative oligonucleotide mixture has the sequence 5′GAATTGTAATACGACTCACTATAGGGNNNNNN 3′ (SEQ ID: 01) where NNNNNN represents the random hexamer region. The random region is preferably a hexamer but in some embodiments it may be 7, 8 or 9 random bases in length or more.

The reagent kit may further include a container containing a reverse transcriptase and a container containing an RNA polymerase. The kit may have a random primer mixture (such as a random hexamer mixture), in addition to the oligonucleotide mixture with a random primer and a promoter region. Additional components may include labeling and fragmentation reagents, nucleotides, a concentrated buffer solution and water.

In a preferred embodiment, the kit includes a collection of at least 1000, 5000, 10,000 or 50,000 different nucleic acid probes designed to detect sequences representing target RNA transcripts. The nucleic acid probes may be immobilized on a substrate. They are typically designed to at least 5000 different exons or at least 500 splice junctions.

The methods and reagent kits of the invention have extensive applications in biological research, diagnostics, toxicology, drug discovery and other areas.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1 is a schematic showing a preferred embodiment of the methods, employing an oligonucleotide primer that contains a random hexamer region and a T7 promoter region.

This method has two cDNA synthesis steps.

FIG. 2 is a schematic showing an embodiment of WTA that includes a rRNA reduction step and generates primarily single strand sense target.

FIG. 3 is a schematic drawing of methods of fragmenting and labeling DNA using uracil DNA-glycosylase (UDG) and an AP endonuclease.

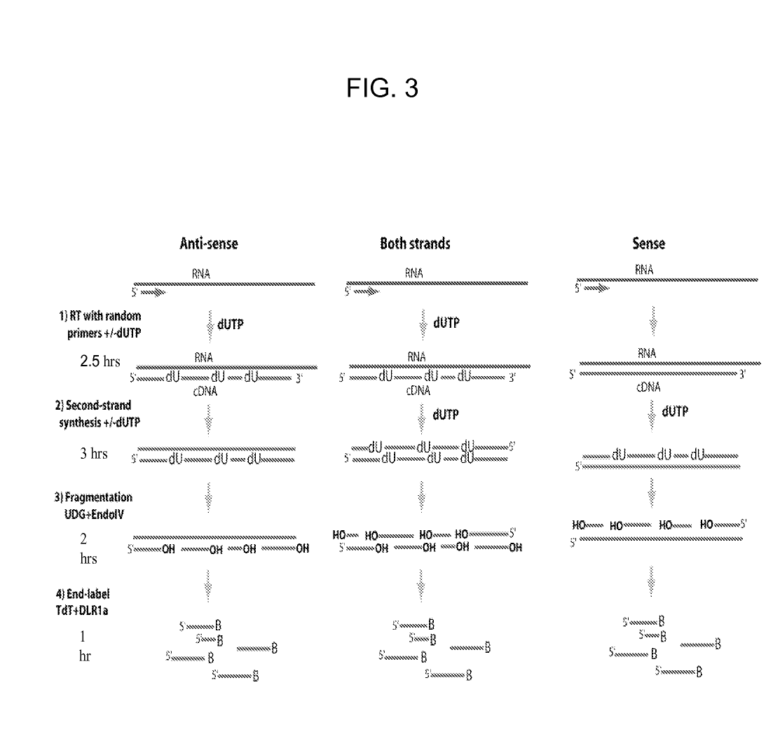

### DETAILED DESCRIPTION OF THE INVENTION

In one aspect of the invention, methods and compositions are provided for analyzing RNA transcription. Methods and compositions for preparing nucleic acid samples that are derived from transcript samples are provided. In preferred embodiments, the nucleic acid samples represent the transcript population in the transcript samples. Therefore, these preferred methods are particularly suitable for preparing nucleic acids samples that are used for interrogating transcript feature/structures such as exons structures and splicing in the transcripts. The methods of the invention generally have a better ability to make transcript anywhere across the target, not just at the 3′ or 5′ end. The preferred methods typically include synthesizing nucleic acids using transcripts as templates and random oligonucleotides as primers (e.g., by reverse transcription reactions). The synthesized nucleic acids are then further processed to obtain nucleic acid samples. The methods are particularly useful for microarray based experiments. However, the sample preparation methods may also be used for other detection methods.

In another aspect of the invention, assay kits that contains one or more primers (which may contain a random region and a fixed content region, such as a T7 promoter), optionally contains a reverse transcriptase, RNA polymerase, labeling reagents, and/or fragmentation reagents.

I. General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GENECHIP. Example arrays are shown on the website at affymetrix.com.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. No. 60/319,253, 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188,and 5,333,675, and each of which is incorporated herein by reference in their entireties for all purposes. The sample may be amplified on the array. See, for example, U.S. Pat. No. 6,300,070 and United States Patent Application 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5, 413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947 and 6,391,592 and U.S. patent application Ser. Nos. 09/916,135, 09/920,491, 09/910,292, and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davis, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Patent Application 60/364,731 and in PCT Application PCT/US99/06097 (published as WO99/47964), each of which also is hereby incorporated by reference in its entirety for all purposes.

The present invention also contemplates depletion of non-target RNAs from total RNA prior to amplification using random primers. Because mRNA may be a minority component of a total RNA sample (less than 2-5%), when analyzing expression it may be desirable to remove RNAs that are non-target components to enrich for target RNAs. One such non-target component of total RNA is ribosomal RNA (rRNA). Methods for depleting rRNA from a nucleic acid sample to enrich for mRNA are disclosed, for example, in U.S. Pat. No. 6,613,516. Kits for depletion of rRNA are available, for example, the RIBOMINUS kit from Invitrogen (PN 1550-01). The RIBOMINUS kit is further described in the instruction manual that is provided with the kit, which is incorporated herein by reference (Version A, 15 Oct. 2004, 25-0752). The kit provides reagents for selective removal of 18S and 28S human and mouse rRNA from total RNA, with an efficiency of about 95% or greater. Briefly, total RNA is hybridized with sequence-specific 5′ biotin labeled oligonucleotide probes to allow formation of complexes between the probes and the rRNA. The rRNA/probe complexes are then removed from the sample using streptavidin coated magnetic beads. The biotinylated probes bind to the streptavidin on the magnetic beads and the magnetic beads can be separated from the supernatant. The supernatant is depleted of rRNA and enriched for non rRNAs, for example, mRNAs. The enriched supernatant may be concentrated. The enriched fraction may contain, for example, mRNA, pre-mRNA, miRNA, siRNA, snRNA, snoRNA, small rRNAs (5S and 5.8S) and tRNA. The RIBOMINUS kit contains 2 probes each for 18S and 28S. The oligonucleotide probes are 18-19 based and have a 5′ biotin label. Each probe contains 5-7 LNA monomers within the oligonucleotide. Other methods for separation of mRNA from rRNA include, for example, isolation of poly-adenylated RNA. LNAs (Locked Nucleic Acids) consist of a ribonucleoside linked between the 2′ oxygen and the 4′ carbon atom of the methylene ring, resulting in an increased melting temperature. Like PNAs, LNAs bind more stably to targets. For more information about LNAs see, Braasch, D. A., and Corey, D. R. (2001), Chem Biol. 1, 1-7, and McTigue, P. M., Peterson, R. J., and Kahn, J. D. (2004), Biochemistry. 43, 5388-5405. See also, Ruan, Y., Le Ber, P., Ng, H., and Liu, E. (2004), Trends Biotechnol. 22, 23-30.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Patent Application 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

The present invention may also make use of the several embodiments of the array or arrays and the processing described in U.S. Pat. Nos. 5,545,531 and 5,874,219. These patents are incorporated herein by reference in their entireties for all purposes.

Additionally, the present invention may have preferred embodiments that include methods for providing genetic information over networks such as the Internet as shown in U.S. patent applications Ser. Nos. 10/063,559, 60/349,546, 60/376,003, 60/394,574, 60/403,381.

### DEFINITIONS

An “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

Nucleic acid library or array is an intentionally created collection of nucleic acids which can be prepared either synthetically or biosynthetically and screened for biological activity in a variety of different formats (e.g., libraries of soluble molecules; and libraries of oligos tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs) as described in U.S. Pat. No. 6,156,501 that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

Complementary: Refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementary exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

Combinatorial Synthesis Strategy: A combinatorial synthesis strategy is an ordered strategy for parallel synthesis of diverse polymer sequences by sequential addition of reagents which may be represented by a reactant matrix and a switch matrix, the product of which is a product matrix. A reactant matrix is a 1 column by m row matrix of the building blocks to be added. The switch matrix is all or a subset of the binary numbers, preferably ordered, between 1 and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

Excitation energy refers to energy used to energize a detectable label for detection, for example illuminating a fluorescent label. Devices for this use include coherent light or non coherent light, such as lasers, UV light, light emitting diodes, an incandescent light source, or any other light or other electromagnetic source of energy having a wavelength in the excitation band of an excitable label, or capable of providing detectable transmitted, reflective, or diffused radiation.

Genome is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

Hybridization conditions will typically include salt concentrations of less than about 1 M, more usually less than about 500 mM and preferably less than about 200 mM. Hybridization temperatures can be as low as 5° C., but are typically greater than 22° C., more typically greater than about 30° C., and preferably in excess of about 37° C. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone.

Hybridizations, e.g., allele-specific probe hybridizations, are generally performed under stringent conditions. For example, conditions where the salt concentration is no more than about 1 Molar (M) and a temperature of at least 25° C., e.g., 750 mM NaCl, 50 mM NaPhosphate, 5 mM EDTA, pH 7.4 (5×SSPE) and a temperature of from about 25° C. to about 30° C.

Hybridizations are usually performed under stringent conditions, for example, at a salt concentration of no more than 1 M and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see, for example, Sambrook, Fritsche and Maniatis. “Molecular Cloning: A laboratory Manual” 2^{nd} Ed. Cold Spring Harbor Press (1989) which is hereby incorporated by reference in its entirety for all purposes above.

The term “hybridization” refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.”

Hybridization probes are oligonucleotides capable of binding in a base-specific manner to a complementary strand of nucleic acid. Such probes include peptide nucleic acids, as described in Nielsen et al., Science 254, 1497-1500 (1991), and other nucleic acid analogs and nucleic acid mimetics. See U.S. Pat. No. 6,156,501.

Hybridizing specifically to: refers to the binding, duplexing, or hybridizing of a molecule substantially to or only to a particular nucleotide sequence or sequences under stringent conditions when that sequence is present in a complex mixture (e.g., total cellular) DNA or RNA.

Isolated nucleic acid is an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

Label for example, a luminescent label, a light scattering label or a radioactive label. Fluorescent labels include, inter alia, the commercially available fluorescein phosphoramidites such as Fluoreprime (Pharmacia), Fluoredite (Millipore) and FAM (ABI). See U.S. Pat. No. 6,287,778.

Ligand: A ligand is a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (e.g., opiates, steroids, etc.), hormone receptors, peptides, enzymes, enzyme substrates, substrate analogs, transition state analogs, cofactors, drugs, proteins, and antibodies.

Linkage disequilibrium or allelic association means the preferential association of a particular allele or genetic marker with a specific allele, or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs more frequently, then alleles a and c are in linkage disequilibrium. Linkage disequilibrium may result from natural selection of certain combination of alleles or because an allele has been introduced into a population too recently to have reached equilibrium with linked alleles.

Microtiter plates are arrays of discrete wells that come in standard formats (96, 384 and 1536 wells) which are used for examination of the physical, chemical or biological characteristics of a quantity of samples in parallel.

Mixed population or complex population: refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but include other undesirable populations. For example, a complex population of nucleic acids may be a sample which has been enriched for desired messenger RNA (mRNA) sequences but still includes some undesired ribosomal RNA sequences (rRNA).

Monomer: refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

mRNA or mRNA transcripts: as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

Nucleic acid library or array is an intentionally created collection of nucleic acids which can be prepared either synthetically or biosynthetically and screened for biological activity in a variety of different formats (e.g., libraries of soluble molecules; and libraries of oligos tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

Nucleic acids according to the present invention may include any polymer or oligomer of pyrimidine and purine bases, preferably cytosine, thymine, and uracil, and adenine and guanine, respectively. See Albert L. Lehninger, Principles of Biochemistry, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

Probe: A probe is a surface-immobilized molecule that can be recognized by a particular target. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

Primer is a single-stranded oligonucleotide capable of acting as a point of initiation for template-directed DNA synthesis under suitable conditions e.g., buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 20, 25, 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

Polymorphism refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

Receptor: A molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety. “Solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

Target: A molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

Reference will now be made in detail to exemplary embodiments of the invention. While the invention will be described in conjunction with the exemplary embodiments, it will be understood that they are not intended to limit the invention to these embodiments. On the contrary, the invention is intended to cover alternatives, modifications and equivalents, which may be included within the spirit and scope of the invention.

Sample Preparation Methods for Whole Transcript Assays

Methods for amplification and analysis of the transcriptome are disclosed. WTA methods are disclosed in combination with methods for fragmentation, labeling and array hybridization. The disclosed WTA methods can be used to perform genome-wide surveys of alternative splicing when combined with exon arrays as disclosed, for example, in U.S. patent application Ser. Nos. 11/036,317 and 11/036,498, both filed Jan. 13, 2005. Methods for amplification are also disclosed in U.S. patent application Ser. Nos. 11/072,136, 10/951,983, 60/542,933 and 60/627,053 which are each incorporated herein by reference in their entireties. Exon arrays may include probes for each exon or predicted exon in a mRNA transcript, instead of just probes to the exons in the 3′ end of the mRNAs or probes to the predicted 3′200 to 600 bases of the mRNA. Unlike oligo dT primed synthesis, which is typically biased toward the 3′ end of mRNAs, the use of random primers to prime first strand cDNA synthesis from the RNA sample results in cDNA production from the entire transcript. However, random priming does not distinguish between amplification of mRNA and amplification of other RNAs in the population, such as ribosomal RNAs (rRNA). In total RNA the mRNA represents about 2% of the total RNA population.

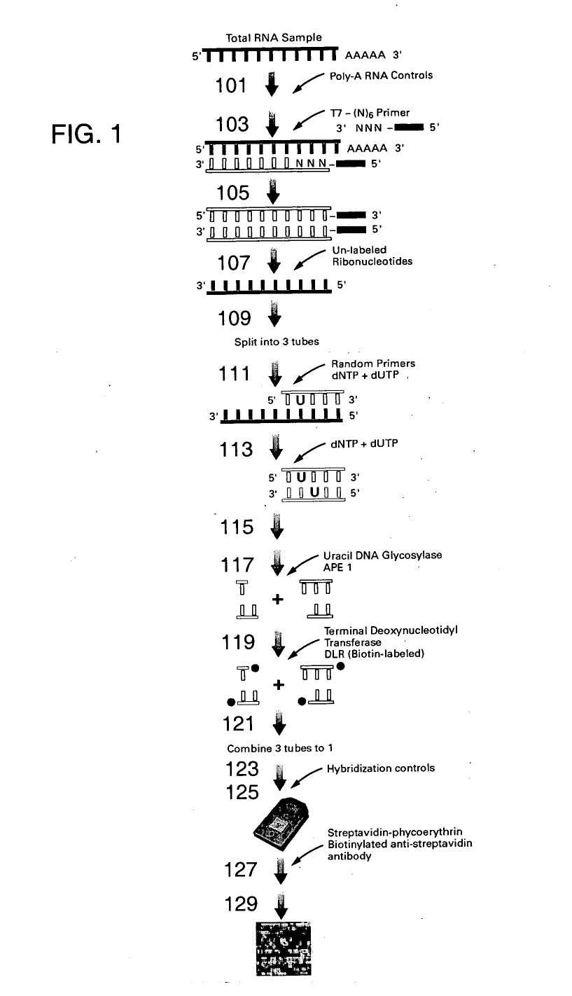

In one embodiment (FIG. 1) the WTA assay includes (1) a first strand cDNA synthesis step at 103, using SuperScript II (Invitrogen) and a random hexamer-T7 promoter primer (Invitrogen), (2) a second strand cDNA synthesis step at 105, using Klenow (exo-) (NEB) and RNase H (Invitrogen), (3) an IVT reaction step at 107, using a MegaScript kit (Ambion), (4) a cRNA purification step at 109 using CleanUp Columns (Qiagen), (5) a second round of first strand cDNA synthesis at 111, using SuperScript II and random hexamers (Invitrogen), (6) a second round of second strand cDNA synthesis at 113 using E. coli DNA polymerase (Invitrogen) and RNase H (Invitrogen), (7) a cDNA purification step at 115 using CleanUp Columns (Qiagen), (8) a fragmentation step at 117 using Uracil DNA glycosylase (UDG) and an AP endonuclease, such as APE 1, and (9) a labeling step at 119 using TdT (Promega) and biotinylated nucleotide, DLR (Affymetrix). A concentration step at 123, hybridization at 125, wash and stain at 127 and scan at 129. Poly A controls may be added at 101 and hybridization controls atl25. In another aspect the fragmentation method may use DNase I digestion instead of UDG and AP endonuclease.

For fragmentation with UDG and APE 1, dUTP is incorporated into the cDNA, the dDNA is treated with UDG to create abasic sites at the uracils, the sample is treated with APE 1 to break phosphodiester bonds at the abasic sites leaving a 3′ OH. The fragments are end labeled at the 3′ end with a biotinylated nucleotide, for example, DLR, using TdT. Polymerases that can be used to incorporate dUTP into DNA include, for example, reverse transcriptase, E. coli DNA polymerase and Taq polymerase. The method is reproducible and fragment size can be controlled, for example, by modifying the amount of uracil incorporated in the cDNA.

In a preferred embodiment the AP endonuclease is human APE 1 or a variant thereof. Human APE 1 is capable of cleaving either single-stranded or double-stranded substrate at AP sites. APE 1 is also known as Hap1, Apex, and Ref1 and can be utilized in conjugation with UDG to perform cleavage at dU incorporation sites in single-strand and double strand DNA. APE 1 is an enzyme of the base excision repair pathway which catalyzes endonucleolytic cleavage immediately 5′ to abasic sites. See Marenstein, DNA Repair 3:527-533 (2004). Additional information about APE1 may be found in Robson, C. N. and Hickson, D. I. (1991) Nucl. Acids Res., 19, 5519-5523, Vidal, A. E. (2001) EMBO J., 20, 6530-6539, Demple, B. et al. (1991) Proc. Natl. Acad. Sci. USA, 88, 11450-11454, Barzilay, G. et al. (1995) Nucl. Acids Res., 23, 1544-1550, Barzilay, G. et al. (1995) Nature Struc. Biol., 2,451-468, Wilson, D. M. III et al. (1995)J. Biol. Chem., 270, 16002-16007, Gorman, M. A. et al (1997) EMBOJ., 16, 6548-6558, Xanthoudakis, S. et al. (1992) EMBO J., 11, 3323-3335, Walker, L. J. et al. (1993) Mol. Cell. Biol., 13, 5370-5376, and Flaherty, D. M. (2001) Am. J. Respir. Cell Mol. Biol., 25, 664-667, each of which is incorporated herein by reference in its entirety for all purposes. Methods of using APE 1 for fragmentation are also disclosed in U.S. Provisional Patent application No. 60/627,053 filed Nov. 12, 2004, which is incorporated herein by reference.

APE1 acts on both dsDNA and ssDNA. The catalytic efficiency of the cleavage of ssDNA is approximately 20-fold less than the activity against AP sites in dsDNA. Catalysis is Mg²⁺ dependent. Unlike the activity of APE1 against AP sites in dsDNA, it does not display product inhibition when acting on an AP site in ssDNA. One unit of APE 1 is defined by the supplier (New England Biolabs) as the amount of enzyme required to cleave 20 pmol of a 34 mer oligonucleotide duplex containing a single AP site in a total reaction volume of 10 μl in 1 hour at 37° C.

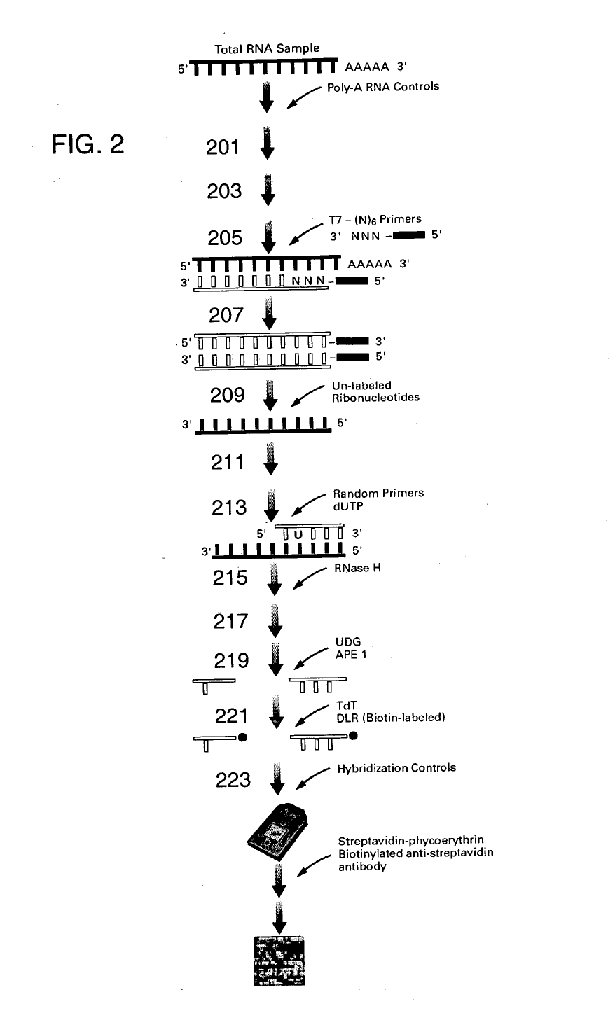

In a preferred aspect WTA methods are optimized to generate amplified and biotinylated sense-strand DNA targets from the entire expressed genome with without bias towards the 3′ end of expression products (FIG. 2). The assay and associated reagents have been optimized for use with the GENECHIP Exon ST Arrays where “ST” stands for “Sense Target”, and the probes have been selected to interrogate regions throughout the entire length of each transcript.

In a preferred aspect methods are disclosed for amplification using as little as 1 μg of total RNA as starting material. In a preferred embodiment the total RNA is first subject to a ribosomal RNA removal procedure where a majority of the 28s and 18s rRNA population is removed from the total RNA sample, minimizing the background signal and increasing the array detection sensitivity and specificity. In preferred aspects more than 50%, more than 75%, more than 90% or more than 95% of the rRNA is removed from the sample.

Following the recommended procedure, from 1 μg of high-quality total RNA, sufficient target is anticipated to be generated to be hybridized to a single 49-format array.

Alternatively, for reduced starting material of 100 ng of total RNA, the rRNA removal process may be omitted. However, the random-priming strategy may introduce higher background signal from the prevalent rRNA molecules resulting in reduced sensitivity and specificity on the arrays.

In a preferred embodiment the assay is optimized to produce sense strand target. The array that the labeled target is hybridized to preferably includes probes that are complementary to sense strand target and omits probes to antisense strand target, except as controls. In another embodiment of the WTA assay the final second strand cDNA synthesis step is omitted so that the cDNA that is fragmented, labeled and hybridized to the array is primarily single stranded cDNA that is sense strand in relation to the starting RNA. In this embodiment the steps of the assay include, (1) an optional rRNA removal step at 201 using RIBOMINUS (Invitrogen), (2) a first strand cDNA synthesis step at 205, using SuperScript II (Invitrogen) and a random hexamer-T7 promoter primer (Invitrogen), (3) a second strand cDNA synthesis step at 207, using DNA Pol I (NEB) and RNases H (Invitrogen), (4) an IVT reaction at 209, using a MegaScript kit (Ambion), (5) a cRNA purification step at 211 using CleanUp Columns (Qiagen), (6) a second round of first strand cDNA synthesis at 213, using SuperScript II and random hexamers (Invitrogen) and RNA hydrolysis at 215, (7) a cDNA purification step at 217 using CleanUp Columns (Qiagen), (8) a fragmentation step at 219 using UDG and APE 1 and (9) a labeling step at 221 using TdT (Promega) and DLR (Affymetrix). The use of DNA Polymerase I for second strand synthesis instead of Klenow (exo-) in the first round was observed to improve strand bias toward the first strand relative to the second strand and to increase the amount of single-stranded final product relative to double-stranded final product. Also, strand bias toward the first strand cDNA was increased by the addition of higher quantities of APE 1 for fragmentation. APE 1 may be used, for example, at 400 U/μg of cDNA. Lower amounts of APE 1 may also be used, for example, 14 U/μg cDNA, but using lower amounts may result in a reduced ability to detect some targets. APE 1 may also be used at 200 to 300 or 300 to 500 units per μg DNA. The rRNA reduction step is preferably used with total RNA samples of at least 1 μg. If the starting sample is between 100 ng and 1 μg of total RNA the rRNA reduction step is preferably omitted.

The fragment size may be controlled by the ratio of dUTP:dTTP in the synthesis reaction. When the ratio is 1:3 the peak of the fragments is at about 55 bases, 1:5 has a peak at about 65 bases and 1:8 has a peak at about 80 bases. For expression analysis of human RNA, 1:3 and 1:5 are preferred ratios for dUTP:dTTP. The assay is compatible with a double stranded or single stranded assay and is compatible with labeling with TdT and DLR.

Kits for performing WTA are also disclosed. The kit in one embodiment is provided in packaging so that the reagents for WT cDNA synthesis are in a first compartment (T7-N₆ primer at 2.5 μg/μl, 5×1^{st} strand buffer, DTT at 100 mM, 10 mM dNTP mix, 40 U/μl RNase inhibitor, SuperScript II at 200 U/μl, 1M MgCl₂, 10 U/μl DNA Pol 1, 3 μg/μl random primers, 2 U/μl RNase H, 10 mM dNTP+dUTP and nuclease free water) and reagents for WT cDNA amplification (NTP mix, 10×IVT buffer, IVT Enzyme mix and IVT control) are provided in a separate second compartment. The reagents for fragmentation and labeling may be provided in a third compartment (cDNA fragmentation buffer, UDG at 10 U/μl, APE 1 at 1000 U/μl, water, TdT, 5×TdT buffer, DLR at 5.0 mM and controls. In one aspect the first and second compartments are packaged together and provided to the user in the as a single unit while the third compartment is provided as a physically separate unit. The modularity of the kit may be used to provide flexibility to the user and to allow the user to store the reagents for a given step together but separated from the reagents used for other steps. In one embodiment the compartments are paper or cardboard boxes.

In one aspect of the invention, methods that are suitable for preparing nucleic acid samples that represent at least 70%, 80%, 90% of the exons of transcripts, or whole transcripts, are provided. In preferred embodiments, the methods are used to prepare nucleic acid samples from at least 70%, 80%, 90% or all exons in a transcript for hybridization with a nucleic acid probe array, such as a high density oligonucleotide array that may contain probes targeting the exons and optionally junctions between exons. The methods of the invention are also particularly suitable for use with tiling arrays such as those described in U.S. patent application Ser. No. 10/815,333, which is incorporated herein. In preferred embodiments, the arrays may have probes that target at least 50%, 70%, 80%, 90% or all the exons of at least 500, 1000, 10,000 transcripts.

In a preferred embodiment, RNA transcript samples (illustrated in FIG. 1) are used as templates for a reverse transcription reaction to synthesize cDNA. Methods for synthesizing cDNAs are well known in the art. In the preferred embodiments, however, a oligonucleotide primer with a random region and a fixed content region may be used. One exemplary primer is a random hexamer and a T7 promoter that may be useful for later in vitro transcription reactions:

(SEQ ID NO.: 1) 5′GAATTGTAATACGACTCACTATAGGGNNNNNN 3′ where NNNNNN represents the random hexamer region).

The random region is useful for random priming of the transcript sequences so that the resulting cDNA is more representative of the various regions of the transcripts than if oligo dT is used to prime first strand cDNA synthesis. In preferred embodiments, the random region of the primer may be 5,6,7,8,9 bases in length. The fixed content region is typically used to provide a desired function in subsequent reactions. For example, a T7 promoter may be useful for an in vitro transcription reaction. One of skill in the art would appreciate that promoters other than T7, such as T3 and SP6 are also commonly used for in vitro transcription and are suitable for use as the fixed content region. Polymerase for various in vitro transcription promoters are commercially available from, for example, Ambion, Inc. (Austin, Tex., USA).

As FIG. 1 shows, the resulting cDNA (typically double stranded) may be used as templates for in vitro transcription reactions to synthesize cRNA. The cRNA targets may be labeled/fragmented for hybridization and detection. However, in a particularly preferred embodiment, the cRNAs are used as templates for another cDNA synthesis reaction using, for example, a random primer. The resulting cDNA may be labeled and fragmented for hybridization and detection. This approach typically enhances the detection sensitivity.

One of skill in the art would appreciate that the invention is not limited to any specific labeling or fragmentation methods. Many suitable labeling and fragmentation methods may be used. Additional DNA fragmentation methods that are suitable for use to enhance hybridization are described in, for example, U.S. Provisional Application Ser. Nos. 60/589,648, 60/545,417, 60/512,569, 60/506,697, all incorporated herein by reference.

The following is a detailed protocol as a non limiting example to illustrate one embodiment. This exemplary protocol was used to detect transcription features, such as exons, alternative splicing, etc., in several large scale experiments with excellent results (data not shown). Exemplary reagents and materials that may be used in the protocol are as follows: RIBOMINUS Human/Mouse Transcriptome Isolation kit (Invitrogen, P/N K1550-01), MAGNA-SEP Magnetic Particle Separator (Invitrogen P/N K1585-01), Betaine, 5M (Sigma P/N B-0300), DEPC treated water, 4 L (Ambion, P/N 9920), SuperScript II, 200 U/μL, 40,000 U, 5× First strand buffer and 0.1 M DTT included (Invitrogen, P/N 18064-071), dNTP mix, 10 mM, 100 μL (Invitrogen, P/N 18427-013), Superase In, 20 U/μL, 2,500 U (Ambion, P/N 26964), Klenow Fragment (3′->5′ exo-), 5U/μL, 1000 U (NEB, P/N M0212L), Magnesium Chloride, 25 mM ( from PCR kit) (ABI) Random Primer, 3 μg/μL, 300 μg (Invitrogen, P/N 48190-011), RNase H, 2 U/μL, 120 U (Invitrogen, P/N 18021-071), Large Fragment of DNA Polymerase I, 3-9 U/μL, 500 U (Invitrogen, P/N 18012-039), DNase I, 1U/μL 5,000 μL (Promega, P/N M6101), One-Phor-All plus Buffer, 10× (Amersham, P/N 27-0901-02), MEGAscript T7 Kit (Ambion, P/N 1334), RNeasy Mini Kit (Qiagen, 74104), QIAquick PCR Purification kit (50) (Qiagen, 28104), Terminal Transferase, recombinant with 5× Buffer and 25 mM CoCl₂ included (Roche Diagnostics, 3 333 574) and DLR-1a, 5 mM (Affymetrix, 900430).

cRNA Amplification

Step 1. First Strand cDNA synthesis

Mix total RNA sample and RP-T7 primer thoroughly in a 0.2 mL of PCR tube: 1 μL Total RNA, (10 ng-100 ng), 1 μL2 pmol/ngRP-T7 primer, and 3 μL H₂O for total volume of 5 μl. Incubate at 65° C. in thermal cycler for 5 minutes, then keep at 4° C. for 2 minutes, and spin down to collect sample. Prepare the RT_Premix_{—}1 as follows: mix 0.5 μL DEPC treated H₂O, 2 μL 5×1^{st} strand buffer, 1 μL 0.1 M DTT, 0.5 μL 10 mM dNTP mix, 0.5 μL 20 U/μL Superase In, and 0.5 μL 200 U/μL SuperScript II for total volume of 5 μl. Add 5 μL of the RT_Premix_{—}1 to the denatured RNA and primer mixture to make a final volume of 10 μL. Mix thoroughly, spin down, and incubate at 25° C. for 10 minutes, at 37° C. for 1 hour, then keep at 4° C. for no longer than 10 minutes.

Step 2. Second Strand cDNA Synthesis

Prepare SS_Premix_{—}1 as follows:4.575 μL DEPC'ed water, 2.8 μL 25 mM MgCl₂, 2.5 μL 5 U/μL Klenow Fragment (exo-), and 0. 125 μL 2 U/μL RNase H, for a total volume of 10 μl. Add 10 μL of the SS_Premix_{—}1 to each first strand reaction to make a final volume of 20 μL. Mix thoroughly and spin down, then incubate at 37° C. for 50 minutes. Inactive the Klenow Fragment (exo-) at 70° C. for 10 minutes, and keep at 4° C. for no longer then 10 minutes to proceed to the next step.

Step 3. IVT for cRNA Amplification Using Ambion MEGAscript T7 Kit

Add the following reagents to the 2nd strand synthesis reaction at room temperature according to the following order:5 μL 75 mM ATP, 5 μL75 mM CTP, 5 μL75 mM GTP, 5 μL 75 mM UTP, 5 μL 10× reaction buffer and 5 μL 10× Enzyme mix, total volume is 50 μl. Mix thoroughly after adding each reagent and spin briefly. Incubate at 37° C. for 16 hours.

Step 4. cRNA Clean-up with RNeasy Columns

Add 50 μL of RNase-free water to the above cRNA product. Follow the RNeasy Mini Protocol for RNA Cleanup handbook from Qiagen that accompanies the RNeasy Mini Kit for cRNA purification. In the last step of cRNA purification, elute the product with 50μ of RNase-free water. Remove 2 μL of the cRNA and add to 78 μL of water to measure the absorbance at 260 nm to determine the cRNA yield. Use speed vacuum to reduce the volume to 7 μL before proceeding to the next step.

Converting cRNA to Double-Stranded cDNA and Labeling

Step 5. Converting cRNA to First Strand cDNA

Mix the cRNA and Random primers thoroughly in a 0.2 mL PCR tube by combining 7 μL cRNA, and 1 μL3 μg/μL Random primers, total volume is 8 μl. Spin briefly and incubating at 70° C. for 5 minutes, at 25° C. for 5 minutes. Prepare RT_Premix_{—}2 by mixing 4 μL5×1^{st} strand buffer, 2 μL0.1 MDTT, 1 μL10 mMdNTP mix, 1 μL20 U/μL Superase In, and 4 μL200U/μL SuperScript II for a total volume of 12 μl. Add 12 μL of the RT_Premix_{—}2 to the denatured RNA and primer mixture to make a final volume of 20 μL. Mix thoroughly and spin briefly. Incubate at 25° C. for 5 minutes, then 37° C. for 1 hour, and keep at 4° C. for no longer then 10 minutes.

Step 6. Second Stranded cDNA Synthesis

Prepare SS-Premix_{—}2 by mixing 9.9 μL DEPC treated water, 5.6 μL 25mM MgCl₂, 4 μL 8.4 U/μL Large Fragment, and 0.5 μL 2 U/μL RNase H, for a total volume of 20 μl. Add 20 μL of the SS_Premix_{—}2 to each first strand reaction to make a final volume of 40 μL. Mix thoroughly and spin down, then incubate at 37° C. for 40 minutes, and keep at 4° C. for no longer than 10 minutes to proceed to the next step or freeze at −20° C.

Step 7. Double-stranded cDNA Clean-up

Follow the QIAquick PCR Purification Kit protocol to clean up the double stranded cDNA. In the last step of double stranded cDNA purification, elute the product with 37 μL of EB Buffer. Remove 2 μL of the cDNA elute and add to 78 μL of water to measure the absorbance at 260 nm to determine the cDNA yield.

Step 8. Double Stranded cDNA Fragmentation

Dilute the 1 U/μL of DNAse I to 0.2 U/μL using 1× One-Phor-All buffer plus. Mix 3.6 μL 10× One-Phor-All buffer plus, 30 μL ds cDNA3 μL and DNAse I (0.2 U/μL). Spin briefly and incubating at 37° C. for 10 minutes and inactivate the DNase I at 95° C. for 10 minutes, then keep at 4° C. Take 1 μL of the fragmented cDNA to check the size with RNA nano kit on Agilent 2100 Bioanalyzer following the kit instruction. The desirable fragment size should be in 50 to 200 bp range. If necessary, use additional DNase I to obtain the desirable size.

Step 9. Fragmented cDNA Labeling:

Prepare the Labeling mix by mixing 14 μL 5×TdT Reaction buffer, 14 μL 25 mM CoCl₂, 1 μL 5 mM DLR-1a, and 4.4 μL Terminal Transferase, (400U/μL) for a total volume of 33.4 μl. Add 33.4 μL of the labeling mix to 35.6 μl of the fragmented cDNA to make a final volume of 69 μL. Mix and spin briefly. Incubate at 37° C. for 60 minutes, and keep at 4° C.

Step 10. Hybridization

Prepare the Hybridization Mix by mixing 100 μL 2×MES Hybridization buffer, 3 μL 3 mM Control Oligo B2, 10 μL 20×RNA control, 2 μL 50mg/μL BSA, acetelated, 2 μL 10 mg/μL Herring sperm DNA, and 14 μL 100% DMSO for a total volume of 131 μl.

Add 131 μL of the Hybridization Mix to 69 μL of the labeling reaction to make a final volume of 200 μL, mix well and denature at 99° C. for 10 minutes and keep at 50° C. for 5 minutes in a thermal cycler. Hybridize the 200 μL of the labeled cDNA to pre-wetted GENECHIP probe array (cDNA test array) at 50° C. for 16 hours. Follow the wash and scan procedures described in the GENECHIP Expression Analysis Technical Manual (Affymetrix, Santa Clara, Calif., USA), incorporated herein by reference.

A second detailed protocol is provided below: the following reagents and materials may be used. Random Primers, 3 μg/μL, Invitrogen Life Technologies, P/N 48190-011, SuperScript II Reverse Transcriptase, (Invitrogen Life Technologies, P/N 18064-071), SUPERASE-IN, (Ambion, P/N 2696), NaOH, 1 N solution, (VWR Scientific Products, P/N MK469360), HCl, 1 N solution, (VWR Scientific Products, P/N MK638860), QIAquick PCR Purification Kit, (QIAGEN, P/N 28104), 10× One-Phor-All Buffer, (Amersham Pharmacia Biotech, P/N 27-0901-02), Deoxyribonuclease I (DNase I), (Amersham Pharmacia Biotech, P/N 27-0514-01), EDTA, 0.5 M pH 8.0, (Invitrogen Life Technologies, P/N 15575-020), Terminal Transferase (including buffer and CoCl₂), 400 U/μL, recombinant, (Roche Applied Science, P/N 3 333 574), and DLR-1a, 5 mM, (Affymetrix, P/N 900430). The starting material for the following protocol is 5 μg of total RNA. Incubations are preferably performed in a thermocycler.

Step 1: cDNA Synthesis

Assemble the RNA/Primer annealing mix as follows. Mix 5 μg Total RNA, 1 μL Random Primer (750 ng/ul) (final 25 ng/μl) and Nuclease-free H₂O to a final volume of 30 μl. Prior to use, dilute Random Primer from 3 μg/μL to 750 ng/pL (1:4 dilution). Incubate the RNA/Primer mix at the following temperatures: 70° C. for 10 minutes and 25° C. for 10 minutes. Chill to 4° C. Prepare the reaction mix for cDNA synthesis. Briefly centrifuge the reaction tube to collect sample at the bottom and add the cDNA synthesis mix to the RNA/primer annealing mix as follows. Mix 30 μLRNA/Primer Annealing Mix, 12 μL 5×1^{st} Strand Buffer (final 1×), 6 μL 100 mM DTT (final 10 mM), 3 μL 10 mM dNTP (final 0.5 mM), 1.5 μL SUPERase-In (20 U/ul) (final 0.5 U/μL), 7.5 μL SuperScript 11 (200 U/ul) (final 25 U/μL) for a total volume of 60 μl. Incubate the reaction at the following temperatures: 25° C. for 10 minutes, 37° C. for 60 minutes and 42° C. for 60 minutes. Inactivate SuperScript II at 70° C. for 10 minutes. Chill to 4° C. To remove RNA add 20 μL of 1 N NaOH and incubate at 65° C. for 30 minutes. Add 20 μL of 1 N HCl to neutralize.

Step 2. Purification and Quantitation of cDNA Synthesis Products

Use QIAquick Column to clean up the cDNA synthesis product (for detailed protocol, see QIAquick PCR Purification Kit Protocols provided by the supplier). Elute the product with 40 μL of EB Buffer (supplied with QIAquick kit). Take 2 ul from above elution and quantify the purified cDNA product by 260 nm absorbance (1.0 A₂₆₀ unit=33 μg/mL of single strand DNA).

Step 3. cDNA Fragmentation

Prepare the reaction mix as follows: mix 4.5 μL10× One Phor-All Buffer, all (˜38 μL) cDNA template (1.5˜5 μg), X μL Dnase I (see note below)(final concentration of 0.6 U/μg of cDNA) and Nuclease-free H₂O to a total reaction volume of 45 μl. Incubate the reaction at 37° C. for 10 minutes. Inactivate DNase I at 98° C. for 10 minutes. The fragmented cDNA is applied directly to the terminal labeling reaction. Alternatively, the material can be stored at −20° C. for later use.

Step 4. Terminal Labeling

Use Roche Terminal Transferase, recombinant with DLR-1a (Affymetrix, Santa Clara, Calif., USA) to label the 3′ termini of the fragment products. Prepare the Terminal Label Reaction by mixing 14 μL 5×TdT Reaction Buffer, 14 μL25 mM CoCl2, 4.375 μL rTDT (400 U/ul), 37 μL cDNA template (1.5-5 ug), 1 μL DLR-1a (5 mM) for final volume of about 70 μl. Incubate the reaction at 37° C. for 60 minutes. Stop the reaction by adding 2 μL of 0.5 M EDTA (PH 8.0). The target is ready to be hybridized onto probe arrays. Alternatively, it may be stored at −20° C. for later use.

Step 5. Target Hybridization

The following Reagents and Materials may be used. 2× MES Hybridization Buffer (See GENECHIP Expression Analysis Technical Manual for preparation). Final 1× concentration is 100 mM MES, 1 M [Na+], 20 mM EDTA, 0.01% Tween-20). For 50 mL: 8.3 mL of 12×MES Stock Buffer, 17.7 mL of 5M NaCl, 4.0 mL of 0.5M EDTA, 0.1 mL of 10% Tween-20, and 19.9 mL of water. Store at 2° C. to 8° C., and shield from light. 12×MES Stock Buffer is 1.22M MES, 0.89M [Na+].

For 1,000 mL mix 64.61 g of MES hydrate, 193.3 g of MES Sodium Salt and 800 mL of Molecular Biology Grade water. Mix and adjust volume to 1,000 mL. The pH should be between 6.5 and 6.7. Filter through a 0.2 μm filter. Preferably the solution is not autoclaved and is stored at 2° C. to 8° C., and shielded from light. If the solution is yellow it may be preferable to discard.

Wash Buffer A: Non-Stringent Wash Buffer is 6×SSPE, 0.01% Tween-20. For 1,000 mL mix 300 mL of 20×SSPE, 1.0 mL of 10% Tween-20 and 699 mL of water. Filter through a 0.2 μm filter. Wash Buffer B: Stringent Wash Buffer is 100 mM MES, 0.1M [Na+] and 0.01% Tween-20. For 1,000 mL mix 83.3 mL of 12×MES Stock Buffer, 5.2 mL of 5M NaCl, 1.0 mL of 10% Tween-20 and 910.5 mL of water. Filter through a 0.2 μm filter and store at 2° C. to 8° C. and shield from light. 2× Stain Buffer for final 1× concentration of 100 mM MES, 1M [Na+], 0.05% Tween-20. For 250 mL mix 41.7 mL of 12×MES Stock Buffer 92.5 mL of 5M NaCl, 2.5 mL of 10% Tween-20 and 113.3 mL of water. Filter through a 0.2 μm filter and store at 2° C. to 8° C. and shield from light. For 10 mg/mL Goat IgG Stock resuspend 50 mg in 5 mL of 150 mM NaCl and store at 4° C. If a larger volume of the 10 mg/mL IgG stock is prepared, aliquot and store at −20° C. until use. After the solution has been thawed it should be stored at 4° C. Avoid additional freezing and thawing if possible. For 100 ml 1× array holding buffer, final concentration 100 mM MES, 1M [Na+] and 0.01% Tween-20, mix 8.3 ml of 12×MES Stock buffer, 18.5 mL 5M NaCl, 0.1 mL 10% Tween-20 and 73.1 mL water. Store at 2° C. to 8° C. and shield from light.

The following reagents may be used: Acetylated Bovine Serum Albumin (BSA) solution, 50 mg/mL, Invitrogen Life Technologies, P/N 15561-020, Herring Sperm DNA, 10 mg/mL, Promega Corporation, P/N D1811, GeneChip Eukaryotic Hybridization Control Kit, Affymetrix, P/N900299, Control Oligo B2, 3 nM, Affymetrix, P/N 900301 (can be ordered separately), 100% DMSO, Sigma, P/N D-4818

Mix the following for each target, scaling up volumes for hybridization to multiple probe arrays. Hybridization Cocktail for Single Midi Probe Array: 100 μL 2×MES Hybridization Buffer (1× final), 3.3 μL Control Oligo B2 (50 pM final), 10 pL20× Spike Controls, 2 μL HS DNA (10 mg/ml)(0.1 mg/ml final), 2 μL Ace-BSA (50 mg/ml) (0.5 mg/ml final), 14 μL 100% DMSO (7% final) and 70 μL Fragmented cDNA for total volume of ˜200 μL.

Equilibrate probe array to room temperature immediately before use. Heat the hybridization cocktail to 99° C. for 5 minutes and hold it at 50° C. Meanwhile, wet the array by filling it with 1× Hybridization Buffer. Incubate the probe array at 50° C. for 10 minutes with rotation. Spin hybridization cocktail at maximum speed to remove any insoluble material. Remove the buffer solution from the probe array and fill with hybridization cocktail. Place probe array in the rotisserie box in 50° C. oven, rotate at 60rpm, and hybridize for 16 hours.

Step 6. Probe Array Wash and Stain

Reagents and Materials that may be used include: 2×MES Stain Buffer (See GeneChip Expression Analysis Technical Manual for preparation), Acetylated Bovine Serum Albumin (BSA) solution, 50 mg/mL, Invitrogen Life Technologies, P/N 15561-020, R-Phycoerythrin Streptavidin, Molecular Probes, P/N S-866, Goat IgG, Reagent Grade, Sigma-Aldrich, P/N I 5256 and Anti-streptavidin antibody (goat), biotinylated, Vector Laboratories, P/N BA-0500.

Prepare SAPE Solution Mix for First and Third Stain by mixing 600.0 μL 2×MES Stain Buffer (1× final), 48.0 μL 50 mg/ml BSA (2 mg/ml final), 12.0 μL1 mg/ml Streptavidin Phycoerythrin (10 μg/ml final), and 540.0 μL DI H₂O, for total volume of 1200 μl. Antibody Solution Mix for Second Stain is as follows: 300.0 μL 2×MES Stain Buffer (1× final), 24.0 μL 50 mg/ml BSA (final 2 mg/ml), 6.0 μL 10 mg/ml Normal Goat IgG (final 0. 1 mg/ml), 6.0 μL 0.5 mg/ml Biotin Anti-streptavidin (final 5 μg/ml), and 264.0 μL DI H₂O for a total volume of 600.0 μL. Follow the instructions described in the GENECHIP Expression Analysis Technical Manual for the washing and staining steps for eukaryotic targets.

In one aspect of the invention, methods for preparing nucleic acid samples that represent RNA transcripts are provided. The methods are particularly suitable for preparing samples that are used for detecting transcript features such as exons and alternative splicing. The methods are suitable for quantitative, semi-quantitative or qualitative detection of such transcript features. The methods can be used to monitor a large number of transcripts including all types of variants such as alternative spliced transcripts. The methods are particular suitable for microarray based parallel analysis of a large number of, such as more than 1000, 5000, 10,000, 50,000 different target transcripts or transcript features. As used herein, the term “target transcript” or “target nucleic acid” is used to refer to transcripts or other nucleic acids of interest.

In a preferred embodiment, the method for preparing a nucleic acid sample includes hybridizing a primer mixture with a plurality of RNA transcripts or nucleic acids derived from the RNA transcripts and synthesizing first strand cDNAs complementary to the RNA transcripts and second strand cDNAs complementary to the first strand cDNAs, where the primer mixture contains oligonucleotides with a promoter region and a random sequence primer region; and transcribing RNA initiated from the promoter region to produce the nucleic acid sample. The primer region can be a random hexamer. The promoter is typically a prokaryotic promoter such as a bacteriophage promoter, preferably a T7, T3 or SP6 promoter.

The method can be used to analyze eukaryotic mRNA or other RNAs. Total RNA samples or poly(A)+ enriched samples are all suitable for use with this method. In a particularly preferred method, the resulting cRNA can be used as templates to synthesize second cDNAs. The second cDNA synthesis may be carried out using random primers such as random hexamer.

While the methods of the invention have broad applications and are not limited to any particular detection methods, they are particularly suitable for detecting a large number of, such as more than 1000, 5000, 10,000, 50,000 different transcript features. For example, the second cDNAs may be fragment/labeled and then hybridized with nucleic acids for detection. Oligonucleotide probes are particularly suitable for detecting specific transcript features such as specific exons and/or splice junctions in transcripts. Typically, a collection of at least 5,000, 10,000, 50,000, 100,000 or 500,000 oligonucleotide probes may be used for detection. The nucleic acid probes may be immobilized on a collection of beads or on a single substrate.

In another aspect of the invention, a reagent kit for the preparing nucleic acid samples is provided. An exemplary reagent kit contains a container comprising an oligonucleotide mixture component and instructions for use of the oligonucleotide mixture where the oligonucleotide in the oligonucleotide mixture component comprises a random primer region and a promoter region. One illustrative oligonucleotide mixture has the sequences of: (SEQ ID NO.: 1) 5′ GAATTGTAATACGACTCACTATAGGGNNNNNN 3′ (NNNNNN represents the random hexamer region).

The reagent kit may further include a container containing a reverse transcriptase and a container containing an RNA polymerase. The kit may have a random primer mixture (such as a random hexamer mixture), in addition to the oligonucleotide mixture with a random primer and a promoter region. Additional components may include labeling and fragmentation reagents, nucleotides, etc.

In a preferred embodiment, the kit include a collection of at least 1000, 5000, 10,000 or 50,000 different nucleic acid probes designed to detect sequences representing target RNA transcripts. The nucleic acid probes may be immobilized on a substrate. They are typically designed to at least 5000 different exons and/or at least 500 splice junctions.

The methods and reagent kits of the invention has extensive applications in biological research, diagnostics, toxicology, drug discovery and other areas. In an exemplary embodiment, transcription of individual exons and splice junction structures are monitored in samples treated with drug candidates. The response of transcription features, such as alternative splicing, to the drug treatment may be analyzed to evaluate the drug candidates. The methods and kits of the invention are particularly suitable for such application because the resulting nucleic acids are more representative of the entire transcript rather than being limited to the 3′ or 5′ region of the transcripts.

In another exemplary application, the methods and kits may be used to process tissue samples to obtain nucleic acid samples. The samples are analyzed for alternatively spliced transcripts. It is well known that alternative splicing is often involved in the pathogenesis of certain diseases. By analyzing the alternative splicing events in the tissue sample, diagnostic information can be obtained. The invention will be further illustrated by the following examples.

### EXAMPLES

### Example 1

### DNA Endonuclease Fragmentation and Terminal Labeling (DEFT Labeling)

Reagents and Materials Required: Random Primers, 3 μg/μL, Invitrogen Life Technologies, P/N 48190-011, SuperScript TI Reverse Transcriptase, Invitrogen Life Technologies, P/N 18064-071, SUPERase. In™, Ambion, P/N 2696, QIAquick PCR Purification Kit, QIAGEN, P/N 28104, 10× One-Phor-All Buffer, Amersham Pharmacia Biotech, P/N 27-0901-02, Deoxyribonuclease I (DNase I), Amersham Pharmacia Biotech, P/N 27-0514-01, EDTA, 0.5 M pH 8.0, Invitrogen Life Technologies, P/N 15575-020, Terminal Transferase (including buffer and CoCl2), 400 U/ul, recombinant, Roche Applied Science, P/N 3 333 574, DLR-1a, 5 mM, Affymetrix, P/N 900430, Second-strand cDNA synthesis kit, Invitrogen, dUTP, Roche P/N 1934554, dNTP set P/N 1969064, Uracil DNA Glycosylase, New England Biolabs P/N M0280S, Endonuclease IV, Epicenter special order, quote AFF950-0104-COLE, 10× REC1™ Buffer 1 (10 mM HEPES-KOH, pH 7.4, 100 mM KCl), Trevigen Inc.

Step 1. Double Strand cDNA Target Preparation

First-strand cDNA Synthesis: Random primer (Invitrogen Life Technologies, 3 μg/μl) was diluted to 750 ng/μl. The following mixture for primer annealing was prepared.5 μlTotal RNA (1 μg/μl)(final 5 μg) is mixed with 1 μl Random Primer (750 ng/μl) (final 25 ng/μl) and Nuclease-free H₂O up to a final total reaction volume of 30 μl.

The RNA/Primer mix at 70° C. for 10 minutes and 25° C. for 10 minutes and then chilled to 4° C. The reaction was performed in a thermocycler. The reaction tube was then briefly centrifuged to collect sample at the bottom. The RNA/Primer Annealing Mix (30 μl) was mixed with 12 μλ5×1st Strand Buffer (final 1×), 6 μl 100 mM DTT (final 10 mM), 3 μl 10 mM dNTP+dUTP (final 0.5 mM), 1.5 μl SUPERase.In™ (20 U/μl) (final 0.5 U/μl), and 7.5 μl SuperScript 11 (200 U/μl) (final 25 U/μl) for a final volume of 60 μl. The cDNA synthesis mix was added to the RNA/primer annealing mix. A stock solution of 10 mM dNTP+1dU:3dT (dNTP+dUTP Mix) is prepared by combining 8 μl of dATP, 8 μl of dCTP, 8 μl of dGTP, 6 μl of dTTP and 2 μl of dUTP stock solutions (100 mM concentration) with 48 μl of H₂O.

The reverse transcription reaction was incubated for 10 min 25° C., for 60 minutes at 37° C., for 60 minutes at 42° C. SuperScript TI enzyme was heat inactivated at 70° C. for 10 minutes. The reaction was stopped by chilling to 4° C.

If only the antisense cDNA strand was to be labeled, sample was purified using QiaQuick column prior to second strand cDNA synthesis. However, we have achieved good results by omitting this purification step and carrying the reaction directly into second-strand synthesis. If no additional dUTP was added to second strand synthesis, the dUTP ratio should be inferior or equal to 1dU:6dT.

Second-strand cDNA synthesis. The second-strand cDNA synthesis reaction was prepared by combining the following components on ice:60 μl First-strand cDNA reaction (˜3-5 μg), 30 μl 5× Second strand Buffer, 3 μl 10 mM dNTP mix, (final concentration in reaction of 200 μM each), 1 μl E. coli DNA Ligase (10 U/μl), 4 μl E. coli DNA Polymerase (10 U/μl), 1 μl E. coli RNase H(2 U/ul), and 51 μl H₂O for a total volume of 150 μl. dUTP may be incorporated during second strand synthesis by using the same stock of 10 mM dNTP+1dU:3dT used for first strand synthesis. cDNA containing dUTP in only the antisense strand (incorporated during first strand synthesis) performed significantly better than target containing dU in both strands. The average fragment size can be controlled by titrating dUTP concentration in cDNA synthesis: the average fragment size increases as dUTP concentration decreases.

The reaction mixture was incubated reaction for 2 hours at 16° C. and 2 μl of T4 DNA polymerase was added and incubated at 16° C. for 5 minutes. The reaction was stopped by adding 10 μl 0.5 M EDTA.

Purification and Quantitation of cDNA Synthesis Products. cDNA synthesis products were cleaned using QIAquick Columns (Qiagen). Product was eluted with 40 μL of EB Buffer (supplied with QIAquick purification kit). The cDNA was quantified by 260 nm absorbance on 2 μl of the elution (1.0 A₂₆₀ unit=50 μg/mL of double strand DNA). The typical yield of ds-cDNA was 8-12 μg at a concentration ≧260 ng/μl.

Typical yields of ds cDNA were found to be between 8 and 12 μg. A minimum amount of cDNA is recommended for subsequent procedures to obtain sufficient material for hybridizing on to the array in addition to the material needed to perform necessary quality control experiments.

Step 2. DNA Endonuclease Fragmentation and Terminal Labeling (DEFT).

The following reactions provided extra volume for analysis of fragmentation and labeling efficiency. If desired the reactions may be scaled down to a final volume of 70 μl so that all the entire target can be hybridized to the array.

Two-Step DEFT Labeling Protocol. ds cDNA was fragmented using the following fragmentation reaction: X μl 9-12 μg ds cDNA, 4.8 μl 10× REC1 Buffer, 4.8 μl Uracil DNA Glycosylase (2 U/μl) (final is 0.8 U/μl cDNA), 3.5 μl Endonuclease IV (20 U/μl) (final is ˜6 U/μl cDNA) and Y μl H₂O to a final volume of 48 μl.

The reaction was incubated at 37° C. for 1-2 hours and stopped by heat inactivation to 93° C. for 1 minute. Two μl were removed for fragmentation analysis on a 4-20% acrylamide gel and stained with SYBR Gold. Alternatively, the size of the fragments was analyzed by loading ˜200 ng of the product to Agilent 2100 Bioanalyzer. Fragments distribution peaked between 50-100 nt.

Fragments were terminal labeled using the following protocol. Mix 44 μl ds cDNA template (9-12 μg), 16.8 μl 5×TdT Reaction Buffer, 16.8 μl 25 mM CoCl₂, 5.3 μl rTDT (400 U/μl), and 1.2 μl DLR-1a (Affymetrix, 5 mM). Total volume is 84 μl.

The reaction was incubated at 37° C. for 60 minutes and stopped by addition of 2 μl of 0.5M EDTA (pH 8.0, Invitrogen Life Technologies). Fourteen μl was removed to be analyzed by gel-shift analysis for labeling efficiency. Six μl H₂O (20 μl final volume) was added and DLR excess label was removed with BioSpin prior to the gel-shift analysis. The remaining target (˜70 μl) was used for hybridization to probe arrays.

Alternatively, a one-Step DEFT Labeling protocol may be used. In the one step DEFT labeling protocol, the fragmentation step and the terminal labeling reaction are combined according to the following protocol. 9-12 μg of ds cDNA template in X μl is mixed with 16.8 μl 5×TdT Reaction Buffer (final 1×), 16.8 μl 25 mM CoCl₂ (final 5 mM), 5.3 μl rTDT (400 U/μl) (final 5.8 U/pmol), 4.8 μl Uracil DNA Glycosylase (2 U/μl) (final ˜0.8 U/μl cDNA), 3.5 μl Endonuclease IV (20 U/μl) (final ˜6 U/μl cDNA), 1.2 μl DLR-1a (5 mM) (final 0.07 mM) and H₂O to a final volume of 84 μl.

The reaction was incubated at 37° C. for 2 hours and stopped by the addition of 2 μl of 0.5 M EDTA (pH 8.0). The reaction product was analyzed by gel-shift as mentioned above. The remaining target (70 μl) was hybridized to probe arrays.

Step 3. Target Hybridization.

Hybridization cocktail was prepared for each target by mixing 100 μl 2×MES Hybridization Buffer (Affymetrix), 3.3 μl Control Oligo B2 (Affymetrix) (final concentration of 50 pM), 10 μl 20× Spike Controls, 2 μl Herring Sperm DNA (Promega corporation, 10 mg/ml) (final concentration of 0.1 mg/ml), 2 μl acetylated-B SA (Invitrogene Life Technologies, 50 mg/ml) (0.5 mg/ml final), 14 μl 100% DMSO (Sigma) (7% final) and 70 μl fragmented cDNA for a total volume of about 200 μl.

The probe array was equilibrated to room temperature immediately before use. The hybridization cocktail was heated to 99° C. for 5 minutes and kept at 50° C. before being spun at maximum speed to remove any insoluble material. Meanwhile, the array was equilibrated in 1× Hybridization Buffer at 50° C. for 10 minutes with rotation and then incubated in hybridization cocktail. Probe array was placed in the rotisserie box in 50° C. oven that rotates at 60rpm, and hybridized for 16 hours. Probe array was washed and stained according to the GeneChip Expression Analysis Technical Manual for eukaryotic targets. The array performance using DEFT fragmented 1dU:3dT and 1dU:4dT ds-cDNA is comparable or better than when using the standard protocol using DNase I.

Step 4. Probe Array Wash and Stain

Reagents and Materials Required include: 2×MES Stain Buffer (See GeneChip Expression Analysis Technical Manual for preparation); Acetylated Bovine Serum Albumin (BSA) solution, 50 mg/mL, Invitrogen Life Technologies, P/N 15561-020; R-Phycoerythrin Streptavidin, Molecular Probes, P/N S-866; Goat IgG, Reagent Grade, Sigma Aldrich, P/N I 5256; and Anti-streptavidin antibody (goat), biotinylated, Vector Laboratories, P/N BA-0500.

The staining reagents was prepared by mixing 600.0 μl 2×MES Stain Buffer (1× final), 48.0 μl 50 mg/ml acetylated BSA (2 mg/ml final), 12 μl 1 mg/ml Streptavidin Phycoerythrin (10 μg/ml final) and 540.0 μl DI H₂O for a final total volume of 1200 μl.

The Antibody Solution Mix for Second Stain was prepared by mixing 300.0 μl 2×MES Stain Buffer, 24.0 μl 50 mg/ml BSA, 6.0 μl 10 mg/ml Normal Goat IgG, 6.0 μl 0.5 mg/ml Biotin Anti-streptavidin and 264.0 μl DI H₂O (total volume is 600.0 μl). The final concentrations are 1×MES stain buffer, 2 mg/ml BSA, 0. 1 mg/ml Normal Goat IgG and 5 μg/ml biotin anti-streptavidin.

Probe Arrays were washed and stained according to the instructions described in the GeneChip Expression Analysis Technical Manual for the washing and staining steps for eukaryotic targets.

### Example 2

### WTA Single Stranded Assay with rRNA Reduction (FIG. 2)

I. rRNA Reduction and Preparation of Total RNA with Diluted Poly-A RNA Controls

In the following example a minimum of 1 μg of total RNA as starting material was used, and the concentration is preferably not below 0.31 μg/μL. For example, 1 μg of total RNA is suspended in a maximum of 3.2 μL of solution.

Step A. Preparation of dilutions of poly-A RNA Controls:

This GeneChip Poly-A RNA Control Kit was used for this step. The poly-A RNA controls are provided as a concentrated stock of 4 different transcripts at staggered concentrations. Dilution buffer is supplied with the kit to prepare the appropriate dilutions (first: 1:20, second: 1:50 and third: 1:50). Non-stick RNase-free microfuge tubes were used for all dilutions. For the dilutions add 2 μL of Poly-A RNA Control Stock to 38 μL of Poly-A Control Dil Buffer to make the First Dilution (1:20). Mix and spin to collect the solution at the bottom of the tube. Add 2 μL of the First Dilution to 98 μL of Poly-A Control Dil Buffer to make the Second Dilution (1:50). Mix and spin to collect the solution at the bottom of the tube. Add 2 μL of the Second Dilution to 98 μL of Poly-A Control Dil Buffer to make the Third Dilution (1:50). Mix and spin to collect the solution at the bottom of the tube. Add 2 μL of the Third Dilution to 1 μg of total RNA to make up the Total RNA/Poly-A RNA Controls Mix.

Step B. Preparation of Hybridization Buffer with Betaine:

The RIBOMINUS Human/Mouse Transcriptome Isolation Kit (Invitrogen) was used in this step. Prepare the buffer by mixing these components: 54 μL 5M Betaine and 126 μL Invitrogen Hybridization buffer for a total of 180 μl for each reaction (this includes a 30 μl overfill).

Step C. RIBOMINUS Probe Hybridization:

The RIBOMINUS Human/Mouse Transcriptome Isolation Kit (Invitrogen) was used in this step. In a 0.2 mL strip tube, mix the following components: up to 3.0 μL total RNA/Poly-A RNA Controls Mix (from Step A). 0.8 μL RIBOMINUS Probe, 100 pmol/μL, and 20 μL Hybridization Buffer with Betaine (from Step B) for a total volume of 23.8 μl. If the total RNA is 1 μg/μL. If the total RNA sample is at a lower concentration of between 0.31 μg/μL to 1 μg/μL, then mix the following components: up to 5.2 μL total RNA/Poly-A RNA Controls Mix (from Step A), 0.8 μL RiboMinus Probe, 100 pmol/μand 30 μL Hybridization Buffer with Betaine (from Step B) for a total of 36.0 μl. Flick the tube gently to mix, spin briefly and incubate at 70° C. for 5 minutes in a thermal cycler. Quench the reaction immediately by placing the tube on ice while preparing the magnetic beads.

Step D. Preparation of Beads:

The RIBOMINUS Human/Mouse Transcriptome Isolation Kit (Invitrogen) was used in this step. Completely re-suspend the magnetic beads bottle by flicking it until no deposit is observed at the bottom of the bottle. Pipette 50 μL of beads suspension into a 1.5 mL Non-stick RNase-free tube. Briefly spin and place the tube with the beads suspension on the magnetic stand for ˜1 minute. Gently aspirate, and discard the supernatant. Preferably the beads are not allowed to dry as this may reduce the recovery of RNA.

Washing: First wash: Add 50 μL of RNase-free water to the beads and re-suspend them by flicking the tube. Place the tube on the magnetic stand for ˜1 minute. Gently aspirate, and discard the supernatant. Second wash: Add 50 μL of RNase-free water to the beads; re-suspend them by flicking the tube. Place the tube on the magnetic stand for ˜1 minute. Gently aspirate, and discard the supernatant. Third wash: Add 50 μL of the Hybridization Buffer with Betaine (from Step B) to the beads and re-suspend them by flicking the tube. Place the tube on the magnetic stand for ˜1 minute. Gently aspirate, and discard the supernatant. Re-suspend the beads in 30 μL (or 20 μL, if 30 μL of Hybridization Buffer with Betaine is added in Step C, following Table 4) of Hybridization Buffer with Betaine, and keep them at 37° C. in a heating block for 1-2 minutes.

Step E. rRNA Reduction:

The RIBOMINUS Human/Mouse Transcriptome Isolation Kit (Invitrogen) was used in this step. Transfer the ice-cooled hybridized sample prepared in Step C to the beads prepared in Step D (the total volume ≦56 μL), mix well, and briefly spin. Incubate the tube with the mixture at 37° C. for 10 minutes in a heating block. After 5 minutes of incubation, gently flick-mix the tube. Briefly spin and place the tube in the magnetic stand for 1-2 minutes to obtain the rRNA-probe pellet. Note: The supernatant contains the RIBOMINUS Total RNA/Poly-A RNA Controls Mix. Transfer the supernatant (≦56 μL) to a 1.5 mL Non-stick RNase-free tube, and leave on ice. Wash the beads by re-suspending them in 50 μL of Hybridization Buffer with Betaine, and incubate at 50° C. for 5 minutes. Place the tube in the magnetic stand and carefully aspirate the supernatant. Transfer and combine the wash-supernatant with the supernatant in the tube from Step E4. The total volume of the RIBOMINUS sample is approximately 100 μL.

Step F. Cleanup.

The GENECHIP IVT cRNA Cleanup Kit is used in this step. Proceed to the cleanup procedure using the cRNA Cleanup Spin Columns from the GENECHIP IVT cRNA Cleanup Kit following the following protocol. If not already done, add 20 mL of Ethanol (100%) to the cRNA Wash Buffer supplied in the GENECHIP IVT cRNA Cleanup kit. Add 350 μL of cRNA Binding Buffer to each sample and vortex for 3 seconds. Add 250 μL of 100% ethanol to each reaction and flick the tube to mix. Apply the sample to the IVT cRNA Column sitting in a 2 mL Collection Tube. Centrifuge for 15 seconds at ≧8,000×g. Discard the flow-through. Transfer the IVT cRNA Column to a new 2 mL Collection Tube and add 500 μL of cRNA Wash Buffer and centrifuge for 15 seconds at ≧8,000×g. Discard the flow-through. Wash again with 500 μL of 80% (v/v) ethanol. Centrifuge for 15 seconds at ≧8,000×g and discard the flow-through. Open column cap and spin at ≦25,000×g (maximum speed) for 5 minutes with the cap left open. Transfer the IVT cRNA Column to a new 1.5 mL Collection Tube and add 11 μL of RNase-free Water directly to the membrane. Spin at ≦25,000×g (maximum speed) for 1 minute. The eluted RIBOMINUS Total RNA/Poly-A RNA Controls Mix is ˜9.8 μL.

Step G. Analysis with Bioanalyzer:

Use 1 μL of the purified sample to check its quality by running the Eukaryotic Total RNA Nano Assay in the Bioanalyzer. Please see the Reagent Kit Guide provided with the RNA 6000 Nano LabChip Kit for instructions. Based on the Bioanalyzer results, an average 20% (≧100 ng) recovery can be obtained depending on the tissue.

II. First Cycle Synthesis of cDNA

Step A. Preparation of RIBOMINUS Total RNA/Poly-A RNA Controls/T7-(N)₆ Primers Mix

This Step requires the use of the GENECHIP WT cDNA Synthesis Kit. Dilute the T7-(N)₆ Primers, 2.5 μg/μL stock 1:5 with RNAse-free water to make up a 500 ng/μL working solution. Mix the diluted T7-(N)₆ Primers with the RNA sample by mixing 4 μL RIBOMINUS Total RNA/Poly-A RNA Controls Mix and 1 μL Diluted T7-(N)₆ Primers, 500 ng/μL for a total of 5 μl for each reaction. Flick the tube to mix, spin down the tube, and incubate for 5 minutes at 70° C. Cool the sample for at least 2 minutes at 4° C., and spin down. Place on ice for use in the section below.

Step B. First-Cycle, First-Strand cDNA Synthesis

This Step requires the use of the GeneChip WT cDNA Synthesis Kit. Prepare the First-Cycle, First-Strand Master Mix by mixing 2 μL 5×1^{st} Strand Buffer, 1 μL 01M DTT, 0.5 μL 10 mM dNTP mix, 0.5 μL RNase Inhibitor and 1 μL SuperScript™ II for each reaction. Add 5 μL of the First-Cycle, First-Strand Master Mix to the tube containing the RIBOMINUS Total RNA/Poly-A RNA Controls/T7-(N)₆ Primers Mix, flick-mix, and spin-down. The total reaction volume is 10 μL. Incubate the reaction at 25° C. for 10 min, 42° C. for 1 hour and 70° C. for 10 min. Cool the reaction to 4° C. for at least 2 minutes before immediately continuing to the First-Cycle, Second-Strand cDNA synthesis. Note: Keeping the reaction at 4° C. longer than 10 min may result in reduced cRNA yields.

Step C. First-Cycle, Second-Strand cDNA Synthesis This Step requires the use of the GeneChip WT cDNA Synthesis Kit. Prepare a First-Cycle, Second-Strand Master Mix by mixing 4.0 μL 17.5 mM MgCl₂ (make a fresh diluation each time by mixing 2 μl 1M MgCl₂ with 112 μl nuclease free water), 0.4 μL 10 mM dNTP Mix, 0.6 μLDNA Polymerase I, 0.2 μL RNase H, and 4.8 μL RNase-free water for a total volume of 10 μl for each reaction. Add 10 μL of the First-Cycle, Second-Strand Master Mix to the reaction tube from the First-Strand cDNA Synthesis Reaction for a total reaction volume of 20 μL. Flick-mix the solution, and spin down. Incubate the reaction at: 16° C. for 120 min without heated lid and 75° C. for 10 minutes with heated lid. Cool the sample for at least 2 minutes at 4° C. before proceeding to the next step immediately: First-Cycle, cRNA Synthesis and Cleanup. Note: Keeping the reaction at 4° C. longer than 10 min may result in reduced cRNA yields.

Step D. First-Cycle, cRNA Synthesis and Cleanup

This Step requires the use of the GeneChip WT cDNA Amplification Kit and the GeneChip Sample Cleanup Module. In a separate tube, assemble the IVT Master Mix at room temperature by mixing 5.0 μL10×IVT Buffer, 20.0 μL IVT NTP Mix and 5.0 μL IVT Enzyme Mix for a total volume of 30 μl for each reaction. Transfer 30 μL of the IVT Master Mix to each First-Cycle cDNA Synthesis Reaction sample to a final volume of 50 μL. Flick-mix the solution, and briefly spin in a microfuge. Incubate the reaction for 16 hours at 37° C.

Proceed to the clean-up procedure for cRNA using the cRNA Cleanup Spin Columns from the GeneChip Sample Cleanup Module following the protocol described below. Store the sample at −80° C. if not purifying the cRNA immediately. If not already done, add 20 mL of Ethanol (100%) to the cRNA Wash Buffer supplied in the GeneChip Sample Cleanup Module. Add 50 μL of RNase-free water to each IVT reaction to a final volume of 100 μL. Add 350 μL of cRNA Binding Buffer to each sample and vortex for 3 seconds. Add 250 μL of 100% ethanol to each reaction and flick-mix. Apply the sample to the IVT cRNA Column sitting in a 2 mL Collection Tube. Centrifuge for 15 seconds at ≧8,000×g. Discard the flow-through. Transfer the IVT cRNA Column to a new 2 mL Collection Tube and add 500 μL of cRNA Wash Buffer and centrifuge for 15 seconds at ≧8,000×g. Discard the flow-through. Wash again with 500 μL of 80% (v/v) ethanol. Centrifuge for 15 seconds at ≧8,000×g and discard the flow-through. Open the column cap and spin at ≦25,000×g (maximum speed) for 5 minutes with the caps open. Transfer the IVT cRNA Column to a new 1.5 mL Collection Tube and add 12 μL of RNase-free water directly to the membrane. Spin at ≦25,000×g (maximum speed) for 1 minute.

The eluted cRNA is ˜10.5 μL. Determine the cRNA yield by spectrophotometric UV measurement at 260 nm, 280 nm and 320 nm: Concentration of cRNA (μg/μL)=[A₂₆₀−A₃₂₀]×0.04× dilution factor; μg cRNA=eluate in μL ×cRNA in μg/μL. Each tube should have an average of ≧10 μg of cRNA. Note: This average yield range may vary depending on the type of tissue used.

III. Second Cycle cDNA Synthesis

Step E. Second-Cycle, First-Strand cDNA Synthesis

This Step requires the use of the GeneChip WT cDNA Synthesis Kit. Mix cRNA with the Random Primers in a strip tube by mixing 8 μg cRNA, 1.5 μL Random Primers (3 μg/μL) and Nuclease-free water to a total final volume of 8 μL. Flick-mix, and spin down the tubes. Incubate the Second-Cycle, cRNA/Random Primers Mix at: 70° C. for 5 minutes and 25° C. for 5 minutes. Cool the samples at 4° C. for at least 2 minutes.

In a separate tube, prepare the Second-Cycle, First-Strand cDNA Synthesis Master Mix by mixing 4.0 μL 5×1^{st} Strand Buffer, 2.0 μL 0.1M DTT, 1.25 μL10 mM dNTP+dUTP and 4.75 μL SuperScript™ II for each reaction. Transfer 12 μL of the Second-Cycle, First-Strand cDNA Synthesis Master Mix to the Second-Cycle, cRNA/Random Primers Mix for a total reaction volume of 20 μL. Mix thoroughly by gently flicking the tubes a few times and centrifuge briefly. Incubate the reactions at: 25° C. for 10 minutes, 42° C. for 90 minutes and 4° C. for at least 2 min.

Step F. Hydrolysis of cRNA and Cleanup of Single-Stranded DNA:

This Step requires the use of the GeneChip Sample Cleanup Module. Add 1 μL of RNase H, 2 U/μL to each of the samples and incubate at: 37° C. for 45 minutes, 95° C. for 5 minutes and 4° C. for 2 minutes. Proceed to the Single-Stranded DNA clean-up using the cDNA Cleanup Spin Columns from the GeneChip Sample Cleanup Module following the protocol as described below. Store the sample at −80° C. if not purifying the Single-Stranded DNA immediately. If not already done, add 24 mL of Ethanol (100%) to the cDNA Wash Buffer supplied in the GeneChip Sample Cleanup Module. Add 80 μL of RNase-free water to each sample, 370 μL of cDNA Binding Buffer, and vortex for 3 seconds. Apply the entire sample (the total volume is 471 μL) to a cDNA Spin Column sitting in a 2 mL Collection Tube. Spin at ≧8,000×g for 1 minute. Discard the flow-through. Transfer the cDNA Spin Column to a new 2 mL Collection Tube and add 750 μL of cDNA Wash Buffer to the column. Spin at ≧8,000×g for 1 minute and discard the flow-through. Open cap of the cDNA Spin Column, and spin at ≦25,000×g for 5 minutes with the caps open. Discard the flow-through, and place the column in a 1.5 mL collection tube. Pipette 15 μL of the cDNA Elution Buffer directly to the column membrane and incubate at room temperature for 1 minute. Then, spin at ≦25,000×g for 1 minute. Pipette again 15 μL of the cDNA Elution Buffer directly to the column membrane and incubate at room temperature for 1 minute. Then, spin at ≦25,000×g for 1 minute. The eluted Single-Stranded DNA is ˜28 μL total. Take 2 μL from each sample to determine the yield by spectrophotometric UV measurement at 260 nm, 280 nm and 320 nm: Concentration of Single-Stranded cDNA (μg/μL) =[A₂₆₀−A₃₂₀]×0.033× dilution factor; μg DNA=eluate in μL ×DNA in μg/μL. Each tube should have ≧5 μg of Single-Stranded DNA.

Step G. Fragmentation of Single-Stranded DNA

This Step requires the use of the GeneChip WT Terminal Labeling Kit. Fragment the samples by mixing 5 μg Single-Stranded DNA, 4.8 μL 10×cDNA Fragmentation Buffer, 1.0 μL of 10 U/μL UDG, 2.0 μL APE 1, 1,000 U/μL and RNase-free Water to a final volume of 48 μl per reaction. Add the above mix to the samples, flick-mix, and spin down the tubes. Incubate the reactions at: 37° C. for 1 hour, 93° C. for 2 minutes, 4° C. for at least 2 min. Flick-mix, spin down the tubes, and transfer 45 μL of the sample to a new tube. The remainder of the sample is to be used for fragmentation analysis using a Bioanalyzer. See the Reagent Kit Guide that comes with the RNA 6000 Nano LabChip Kit for instructions. The range in peak size of the fragmented samples should be approximately 55-65 bp. If not labeling the samples immediately, store the fragmented Single-Stranded DNA at −20° C.

Step H. Labeling of Fragmented Single-Stranded DNA:

Prepare the labeling reactions by mixing 45 μL Fragmented Single-Stranded DNA, 12 μL 5×TdT Buffer, 2 μL TdT, and 1 μL 5 mM DNA Labeling Reagent for a total volume of 60 μl for each reaction. Add 15 μL of the Single-Stranded DNA Fragmentation Master Mix to the DNA samples, flick-mix, and spin them down. Incubate the reactions at: 37° C. for 60 min., 70° C. for 10 minutes and 4° C. for at least 2 min. Optionally remove 4 μL of each sample for Gel-shift analysis.

III Hybridization of Labeled Target on the Arrays

Prepare the Hybridization Cocktail in a 1.5 mL RNase-free microfuge tube by mixing 10 μL 20× Eukaryotic Hybridization Controls (bioB, bioC, bioD, cre) for 1.5, 5, 25 and 100 pM, respectively 60 μl fragmented and labeled DNA target (approximately 5 μg), 2 μL Herring Sperm DNA (10 mg/mL) for final concentration of 0.1 mg/mL, 3.3 μl Control Oligonucleotide B2 for final concentration of 50 pM, 2 μL Acetylated BSA (50 mg/mL) for final of 0.5 mg/μL, 100 μL 2× Hybridization Buffer, 14 μL DMSO (7% final), and RNase free H₂O up to a final volume of 200.0 μL. If a portion of the sample was set aside for gel-shift analysis the sample volume is 56 μl.

Flick-mix, and centrifuge the tube. Heat the Hybridization Cocktail at 99° C. for 5 min. Cool to 45° C. for 5 minutes, and centrifuge at maximum speed for 1 minute. Label the Exon 1.0 ST array for the sample that will be run. Inject ˜200 μL of the specific sample into the array through one of the septa. Place array in 45° C. hybridization oven, at 60 rpm, and incubate for about 16 hours.

IV. Array Wash, Stain, and Scan

Use the fluidics protocol MES_EukGE-WS2v5-450-DEV for wash and stain procedures as described in the GeneChip Expression Analysis Technical Manual (Section 2: Eukaryotic Sample and Array Processing), with the following change: When adding staining reagents to the FS-450 fluidics station, place one vial containing Streptavidin Phycoerythrin (SAPE) stain solution mix in sample holder 1. Place one vial containing anti-streptavidin biotinylated antibody stain solution in sample holder. Place one vial containing 800 μL Array Holding Buffer in sample holder 3. Proceed with wash, stain procedure as detailed in the Manual. If a FS-450 instrument is unavailable, proceed with washing and staining with fluidics protocol EukGE-WS2v5_{—}400 and add holding buffer to the cartridge manually prior to scanning.

Scan the probe array according to the GeneChip Expression Analysis Technical Manual (Section 2: Eukaryotic Sample and Array Processing). This takes about 30 minutes per array. Generate .CHP files with PLIER algorithm.

It is to be understood that the above description is intended to be illustrative and not restrictive. Many variations of the invention will be apparent to those of skill in the art upon reviewing the above description. All cited references, including patent and non-patent literature, are incorporated herein by reference in their entireties for all purposes.

What is claimed is:

1. A method for analyzing a plurality of transcripts comprising:
a) obtaining a first sample comprising target and non-target RNA;
b) removing at least some of the non-target RNA from the first sample to generate a second sample, wherein the ratio of target RNA to non-target RNA is higher in the second sample than in the first sample;
c) hybridizing a primer mixture with target RNA transcripts in the second sample or nucleic acids derived from target RNA transcripts in the second sample; synthesizing first strand cDNAs complementary to the target RNA transcripts and second strand cDNAs complementary to the first strand cDNAs, to produce first cDNAs, wherein the primer mixture comprises oligonucleotides comprising an RNA polymerase promoter region and a random sequence primer region;
d) transcribing RNA initiated from the promoter region to produce cRNAs;
e) hybridizing a random primer mixture with the cRNAs;
f) synthesizing second cDNAs from the random primers in the presence of at least one modified DNA precursor nucleotide substrate for a DNA glycosylase.
g) fragmenting the second cDNAs to produce fragmented cDNAs; and
h) hybridizing fragmented cDNAs with a plurality of nucleic acid probes to detect the nucleic acids representing target transcripts.

2. The method of claim 1 wherein step (b) includes the steps of incubating the first sample with an oligonucleotide mixture comprising at least one oligonucleotide attached to a solid support, wherein the oligonucleotide is complementary to a non-target RNA, to allow complexes to form between said oligonucleotide and said non-target RNA and removing said complexes from the sample.

3. The method of claim 2 wherein the oligonucleotide comprises LNAs.

4. The method of claim 2 wherein the oligonucleotide comprises PNAs.

5. The method of claim 1 or 2 wherein at least one of the non-target RNAs is a ribosomal RNA.

6. The method of claim 2 wherein the oligonucleotide mixture comprises a plurality of oligonucleotide sequences attached to one or more solid supports, wherein the oligonucleotides are complementary to ribosomal RNA.

7. The method of claim 6 wherein each of the oligonucleotides comprises LNAs or PNAs.

8. The method of claim 1 wherein the polymerase used for second strand synthesis in step (c) is DNA polymerase I.

9. The method of claim 1 wherein the polymerase used for second strand cDNA synthesis in step (c) is Klenow (exo minus).

10. The method of claim 1 wherein the polymerase used for second strand cDNA synthesis in step (c) is E. coli DNA Polymerase I.

11. The method of claim 1 wherein the second cDNA is fragmented in a reaction mixture comprising APE 1.

12. The method of claim 6 where the reaction mixture comprises at least 350 units of APE 1 per microgram of cDNA.

13. The method of claim 6 wherein the reaction mixture comprises between 200 and 500 units of APE 1 per microgram of cDNA.

14. The method of claim 1 wherein the sample is at least 1 μg of total RNA.

15. The method of claim 1 wherein the non-target RNA is ribosomal RNA.

16. The method according to claim 1 wherein the modified DNA precursor is dUTP.

17. The method of claim 1 wherein the step of fragmenting is by means of excising the modified DNA precursor with a Uracil DNA Glycosylase (UDG) to generate abasic sites and cleaving at the abasic sites with an endonuclease.

18. The method according to claim 17 wherein the endonuclease is endonuclease Ape 1.

19. The method according to claim 1 wherein the modified DNA precursor partially replaces a normal precursor nucleotide.

20. The method according to claim 19 wherein the modified DNA precursor is dUTP and the normal precursor nucleotide is dTTP.

21. The method according to claim 20 wherein the ratio of dUTP to dTTP added is about 1 to 3 to about 1 to 5.

22. The method according to claim 20 wherein dUTP is incorporated into single stranded cDNA during reverse transcription.

23. The method according to claim 20 wherein dUTP is incorporated in a single strand.

24. The method according to claim 20 wherein dUTP is incorporated in a sense strand.

25. The method according to claim 20 wherein dUTP is incorporated in an antisense strand.

26. The method according to claim 20 wherein dUTP is incorporated in both sense and antisense strands of the double stranded cDNA.

27. A method for analyzing a plurality of transcripts comprising:
a) obtaining a total RNA sample wherein said sample has between 100 ng and 1 μg of total RNA;
b) hybridizing a primer mixture with target RNA transcripts in the sample or nucleic acids derived from target RNA transcripts in the sample; synthesizing first strand cDNAs complementary to the target RNA transcripts, wherein the primer mixture comprises oligonucleotides comprising an RNA polymerase promoter region and a random sequence primer region;
c) synthesizing second strand cDNAs complementary to the first strand cDNAs using E. coli DNA polymerase 1, to produce first double stranded cDNAs;
d) transcribing RNA initiated from the promoter region to produce cRNAs;
e) hybridizing a random primer mixture with the cRNAs;
f) synthesizing a population of second cDNAs from the random primers in the presence of at least one modified DNA precursor nucleotide substrate for a DNA glycosylase;
g) fragmenting the population of second cDNAs to produce fragmented cDNAs using UDG and APE 1; and
h) hybridizing the fragmented cDNAs with a plurality of nucleic acid probes to detect the nucleic acids representing target transcripts.

28. The method of claim 29 wherein the population of cDNA generated in step (f) is more than 50% sense strand cDNA.

29. The method of claim 29 wherein the population of cDNA generated in step (f) is more than 80% sense strand cDNA.

30. The method of claim 29 wherein the population of cDNA generated in step (f) is more than 95% sense strand cDNA.

31. The method of claim 31 wherein more than 50% of the sense strand cDNA is single stranded.

32. The method of claim 29 wherein the concentration of APE 1 is at least 300 units per μg of cDNA.

33. The method of claim 29 wherein the concentration of APE 1 is at least 400 units per μg of cDNA.


SEQUENCE LISTING (1 sequence)

SEQ ID NO: 1 - DNA, 32 bp, Artificial
  Synthetic oligonucleotide
  misc_feature (27)..(32): n is a, c, g, or t
gaattgtaatacgactcactatagggnnnnnn